(19) United States
(12) Patent Application Publication
(10) Pub. No.: US 2022/0130128 A1
(43) Pub. Date: Apr. 28, 2022

(54) SYSTEM AND METHOD FOR NORMALIZING VOLUMETRIC IMAGING DATA OF A PATIENT

(71) Applicant: Milan Madhavji, Mississauga, Ontario (CA)

(72) Inventor: Milan Madhavji, Mississauga, Ontario (CA)

(21) Appl. No.: 17/419,383

(22) PCT Filed: Jul. 14, 2020
(86) PCT No.: PCT/CA2020/050981

§ 371 Date: Jun. 29, 2021

Related U.S. Application Data
(60) Provisional application No. 62/875,029, filed on Jul. 17, 2019.

(51) Int. Cl. G06T 19/20; A61B 6/14; A61C 9/00; G16H 30/40; G06N 20/00; G06T 15/08; G06T 17/00; G06T 7/00

(52) U.S. Cl. CPC G06T 19/20; A61B 6/14; A61C 9/004; G16H 30/40; G06N 20/00; G06T 15/08; G06T 2200/04; G06T 7/0012; G06T 2210/41; G06T 2219/2021; G06T 2219/2004; G06T 2207/30036; G06T 17/00

(57) ABSTRACT

A method for mapping patient-specific volumetric imaging data includes acquiring volumetric imaging data of an anatomical structure of a patient, imposing the imaging data of the anatomical structure to a three-dimensional reference model to conform at least approximately with the imaging data representing at least a portion of the anatomical structure of the patient to map the volumetric imaging data representing at least a portion of the anatomical structure relative to the at least a portion of the three-dimensional reference model. The normalized volumetric data may be from a plurality of patients. The normalized data may be used as input data for a model or as training data for a machine learning algorithm to train a model for diagnosing a patient condition or determining or evaluating a treatment plan for a patient condition.

### TECHNICAL FIELD

The present application generally relates to a system and method for mapping patient-specific volumetric imaging data and more specifically to a system and method for mapping patient-specific volumetric imaging data from 3D radiographic images on a standardized 3D reference model of an anatomic structure.

### BACKGROUND

Analysis of patient-specific imaging data, such as radiographic images, is commonly performed by medical practitioners in order to provide a basis for diagnosis of patient ailments and to potentially build a treatment plan therefor. In dental radiology, analysis of cephalograms is commonly performed by dentists and orthodontist to study the structure of the hard and soft tissues of the head and in particular of the face, jaw and dentition. Cephalometric analyses can be used to evaluate the dentition of a patient and provide valuable information for recognizing anatomic abnormalities or anomalies, to predict future changes in the patient, and to study the effects of current treatment plans.

A cephalometric radiograph or cephalogram is an x-ray radiograph of the head wherein a cephalometer is used to obtain standardized and comparable craniofacial images on radiographic films. A cephalometric radiograph may be generated from a plurality of individual x-ray images which may be gathered by relative movement between an x-ray imaging apparatus and the patient so as to acquire a plurality of views of the body structures of the specific patient. The plurality of views may then be processed to yield one or more two-dimensional representations. These images may then be examined by human experts, technicians, transmitted for remote analysis, or for computerized post-processing. Such computerized post-processing may include, for example, the assembly of the two-dimensional representations to construct a three-dimensional representation of the body structure of the patient.

Some features in a cephalogram are clinically relevant and may be identified by way of landmarking or annotation by a human expert. In some cephalograms, many such points may be identified. This may be performed manually or may be performed using suitable computer software. The cephalometric landmarks are used as reference points for the construction of various cephalometric lines or planes and for subsequent numerical determination of cephalometric analysis measurements. The geometric relationship between the cephalometric landmarks may be relied upon to make clinical diagnoses or treatment plans for the patient.

Analysis of radiographic images to identify landmarks or make annotations typically requires input from a skilled person and often requires a substantial time investment. Moreover, such analysis is typically performed on a case-by-case basis and is largely dependent on the knowledge and skill of the expert as well as the quality of the patient specific data. Accordingly, analytical results and subsequent diagnoses and treatment plans determined or evaluated therefrom can be inconsistent.

Automation of the annotation process has been contemplated in the past in order to accelerate the greater process of identifying patient-specific anatomical anomalies and determining a diagnosis and/or a treatment plan for addressing such anomalies. However, automation of the step of annotation of radiographic images is complicated by differences in patient anatomy and inhomogeneity between cephalometric images. Moreover, such systems continue to rely upon two-dimensional cephalometric images for analysis and making annotations. Accordingly, not only do such systems continue to rely on expert analysis to determine the clinical relevance of patient-specific anatomical image data, but such systems do not provide a direct translation of annotations into the three-dimensional context of the patient-specific image data. Accordingly, such systems do not permit anatomically relatable positioning of the landmarks or clinically relevant points of interest.

It is desirable to provide a means by which the clinical relevance of patient-specific anatomical data can be determined with reduced expert input and whereby clinically relevant landmarks, annotations or points of interest in the three-dimensional patient-specific image data and diagnostic findings may be used for determining a suitable patient-specific diagnosis or treatment plan, preferably automatically.

### SUMMARY

The present application generally relates to a system and method for mapping patient-specific volumetric imaging data and more specifically to a system and method for mapping patient-specific volumetric imaging data from 3D radiographic images on a standardized 3D reference model of an anatomic structure.

One general aspect includes a method for mapping patient-specific volumetric imaging data relative to a three-dimensional reference model. The method includes the steps of acquiring volumetric imaging data of an anatomical structure of at least one patient; imposing the volumetric imaging data of the anatomical structure to the three-dimensional reference model of the anatomical structure; and, deforming at least a portion of the three-dimensional reference model to conform at least approximately with the volumetric imaging data representing at least a portion of the anatomical structure of the at least one patient to map volumetric imaging data representing at least a portion of the anatomical structure of the at least one patient relative to the at least a portion of the three-dimensional reference model. The at least one anatomical structure of the at least one patient may be a craniodental structure.

The three-dimensional reference model may include a plurality of vertices and the step of deforming at least a portion of the three-dimensional reference model may further include the step of: changing a position of at least one of the plurality of vertices.

The patient-specific volumetric imaging data may further include at least one annotation and the method may further include the step of mapping a position of the annotation relative to the at least a portion of the three-dimensional reference model. The annotation may be a box bounding a point of interest in the volumetric imaging data of the anatomical structure of the at least one patient. The box may have a width, a depth, a length, a position, and an orientation. At least one of the width, the depth, the length, the position and the orientation of the annotation may be mapped relative to the at least a portion of the three-dimensional reference model.

The method may further include the step of using the mapped volumetric imaging data of the at least a portion of the anatomical structure as at least one of training data and input data for a model. The method may further include the step of determining at least one of a diagnosis and a treatment plan based on the mapped volumetric imaging data. The model may be for diagnosing at least one condition of the at least one patient based on the patient-specific mapped volumetric imaging data. The model may be for at least one of determining and evaluating at least one patient treatment plan based on the patient-specific mapped volumetric imaging data.

In another aspect, the at least one patient may include a plurality of patients and the method may further include the step of using the mapped volumetric imaging data as training data for a machine learning algorithm to train a model. The model may be for diagnosing at least one condition of the at least one patient based on the patient-specific mapped volumetric imaging data. The model may be for at least one of determining and evaluating at least one patient treatment plan based on the patient-specific mapped volumetric imaging data.

Another general aspect includes a system for mapping patient-specific volumetric imaging data relative to a three-dimensional reference model. The system includes an imaging device for acquiring volumetric imaging data of an anatomical structure of at least one patient, means for imposing the volumetric imaging data of the anatomical structure to the three-dimensional reference model of the anatomical structure, and, means for deforming at least a portion of the three-dimensional reference model to conform at least approximately with the volumetric imaging data representing at least a portion of the anatomical structure of the at least one patient to map the volumetric imaging data representing at least a portion of the anatomical structure of the at least one patient relative to the at least a portion of the three-dimensional reference model. The at least one anatomical structure of the at least one patient may be a craniodental structure.

The three-dimensional reference model may include a plurality of vertices. Deforming at least a portion of the three-dimensional reference model may include means for changing the position of at least one of the plurality of vertices.

The patient-specific volumetric imaging data may further include at least one annotation and the system further includes means for mapping a position of the annotation relative to the at least a portion of the three-dimensional reference model. The annotation may be a box bounding a point, object or structure of interest in the volumetric imaging data of the anatomical structure of the at least one patient. The box may have a width, a depth, a length, a position, and an orientation. At least one of the width, the depth, the length, the position and the orientation of the annotation may be mapped relative to the at least a portion of the three-dimensional reference model using suitable means.

The system may further include means for using the mapped volumetric imaging data of the at least a portion of the anatomical structure as at least one of training data and input data for a model. The system may further include means for determining at least one of a diagnosis and a treatment plan based on the mapped volumetric imaging data. The model may be for diagnosing at least one condition of the at least one patient based on the patient-specific mapped volumetric imaging data. The model may be for at least one of determining and evaluating at least one patient treatment plan based on the patient-specific mapped volumetric imaging data.

In another aspect, the at least one patient may include a plurality of patients. The system may include means for using the mapped volumetric imaging data as training data for a machine learning algorithm to train a model. The model may be for diagnosing at least one condition of the at least one patient based on the patient-specific mapped volumetric imaging data. The model may be for at least one of determining and evaluating at least one patient treatment plan based on the patient-specific mapped volumetric imaging data.

A system of one or more computers or computerized architectural components can be configured to perform particular operations or actions by virtue of having software, firmware, hardware, or a combination thereof installed on the system, or accessible to the system via, for example, a remote server, that in operation causes or cause the system to perform the actions. One or more computer programs recorded on one or more computer storage devices can be configured to perform particular operations or actions by virtue of including instructions that, when executed by data processing apparatus, cause the apparatus to perform the actions automatically and/or in real-time.

### BRIEF DESCRIPTION OF THE DRAWINGS

Exemplary non-limiting embodiments are described with reference to the accompanying drawings in which:

FIG. 1 is a flow chart of the method according to one aspect of the present invention;

FIG. 2 is a representation of a three-dimensional reference model as used in the present invention;

FIG. 3 is a representation of patient volumetric imaging data imposed relative to the three-dimensional reference model shown in FIG. 2;

FIG. 4 further illustrates the alignment of the patient volumetric imaging data relative to the three-dimensional reference model shown in FIG. 3;

FIG. 5 illustrates the patient volumetric imaging data annotated in the three-dimensional context of the reference model and patient volumetric imaging data;

FIG. 6 illustrates the data structure of an annotation; and,

FIG. 7 illustrates a three-dimensional visualization of annotations relative to the reference model.

### DETAILED DESCRIPTION

The present application generally relates to a system and method for mapping patient-specific volumetric imaging data and more specifically to a system and method for mapping patient-specific volumetric imaging data from 3D radiographic images on a standardized 3D reference model of an anatomic structure.

Aspects of this disclosure are directed to a system and method for providing analysis of volumetric imaging data of an anatomical structure of a patient, such as the head of a patient or the craniodental region which includes the teeth, jaw and other structures of the cranium relating thereto. It should be understood that the present invention and analysis may be applied using volumetric imaging data of any anatomical structure, such as, for example, the hands or feet, knees or organs.

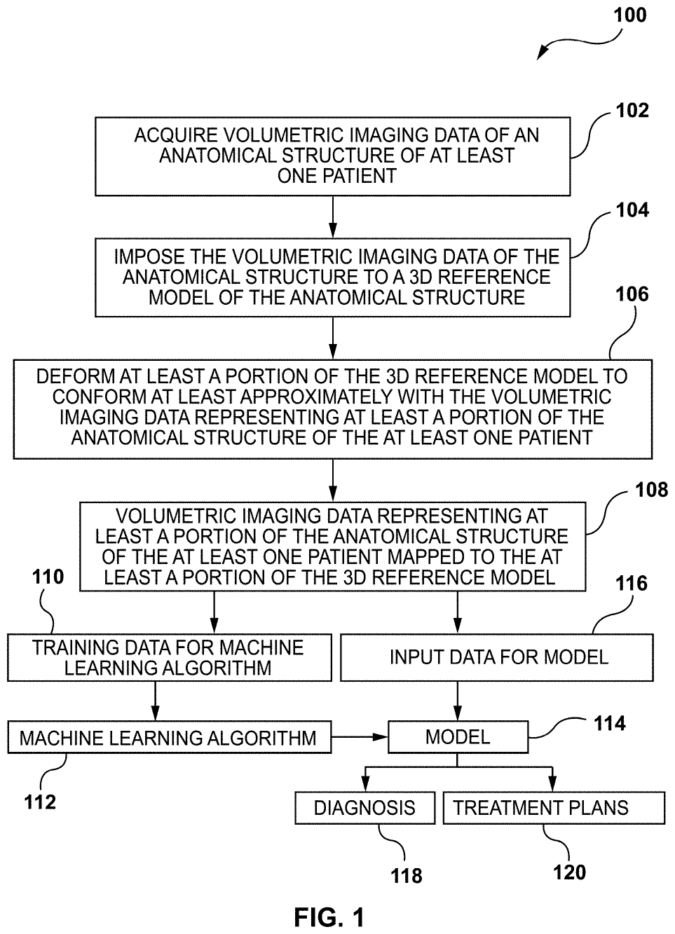

With reference to FIG. 1, there is provided a method 100 for mapping patient-specific volumetric imaging data relative to a three-dimensional reference model according to one aspect of the present invention. At step 102, volumetric imaging data of an anatomical structure of at least one patient is acquired. Then, at step 104, the volumetric imaging data of the anatomical structure is imposed to a three-dimensional reference model of the anatomical structure. At step 106, at least a portion of the three-dimensional reference model is deformed to conform at least approximately with the volumetric imaging data representing at least a portion of the anatomical structure of the at least one patient to provide at step 108 volumetric imaging data representing at least a portion of the anatomical structure of the at least one patient mapped to the at least a portion of the three-dimensional reference model.

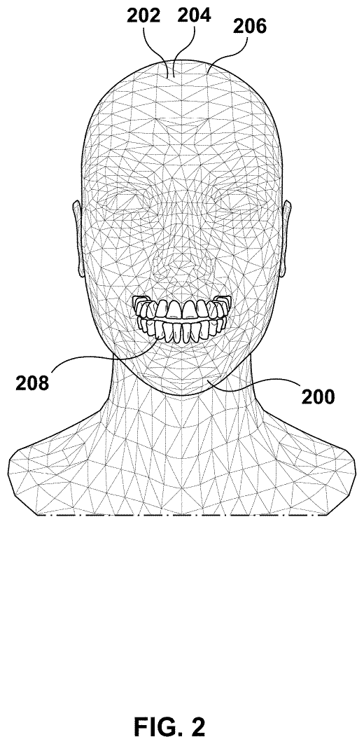

Implementational details of the above-described method are discussed further hereinafter with respect to FIGS. 2 to 5. However, the method 100 may then proceed to step 110 wherein the mapped volumetric imaging data of the at least a portion of the anatomical structure of the at least one patient is used as training data for a machine learning algorithm. The machine learning algorithm may be used to train the model at step 112 to provide at step 114 a trained model. As shown with reference to step 116, the mapped volumetric imaging data of the at least one patient may be used as input data for the model. The model may then produce one or more diagnoses as shown at step 118 or one or more treatment plans as shown at step 120. It should be understood that the model may also provide an evaluation of an existing treatment plan.

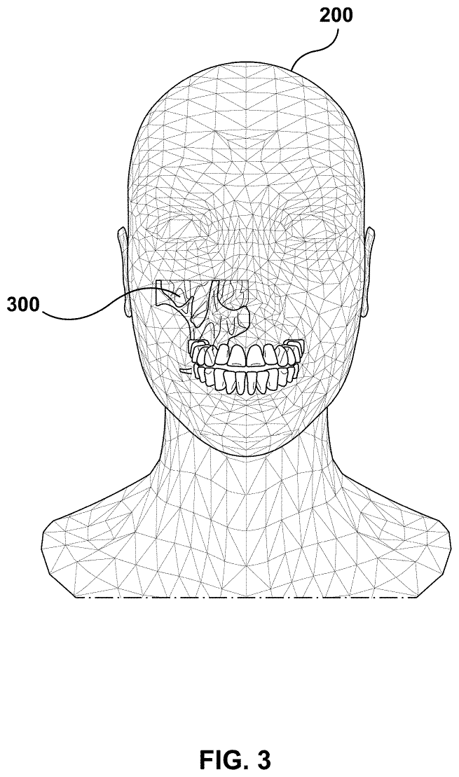

FIG. 2 illustrates an exemplary three-dimensional reference model 200 of an anatomical structure such as that previously described with respect to step 104 of FIG. 1. In the example shown in FIG. 2, the anatomical structure is the head of a patient, including the craniodental region which includes the teeth, jaw and other structures of the cranium relating thereto. The three-dimensional reference model 200 may be formed by way of a plurality of polygons or faces 202 meeting at any number of edges 204 and vertices 206 generally collectively referred to herein as “scaffolding”. As discussed further herein with reference to FIG. 3, FIG. 2 shows the three-dimensional reference model 200 having imposed thereon a portion of volumetric image data representing dentition 208 of a patient. It should be understood that the volumetric image data and the three-dimensional reference model 200 are distinct from one another.

FIG. 3 illustrates volumetric imaging data 300 of a patient imposed or aligned with the three-dimensional reference model 200. The volumetric imaging data 200 of the patient corresponds with at least a portion of the anatomical structure represented by the three-dimensional reference model 200. For example, where the three-dimensional reference model 100 is representative of a cranium, then the volumetric imaging data 200 of the patient is also sourced from the cranium of the patient, but may only represent a portion thereof, such as a craniodental structure. The volumetric imaging data 300 of the patient may be acquired using any suitable scanning technology, such as, for example, computer-aided tomography (CT) scans. In medical imaging, it may be preferred to use a specialized type of CT scan called “cone beam computed tomography”, or CBCT, for short. It should be understood that other types of scanning technology may be used to acquire the volumetric imaging data from the patient.

It should be further understood that patient-specific volumetric imaging data 300 from one or more patients may be stored in a suitable local, remote or cloud-based storage medium and subsequently retrieved or uploaded to a user interface from a using a suitable interface, such as a web browser or other file transfer protocol client or a cloud-based interface.

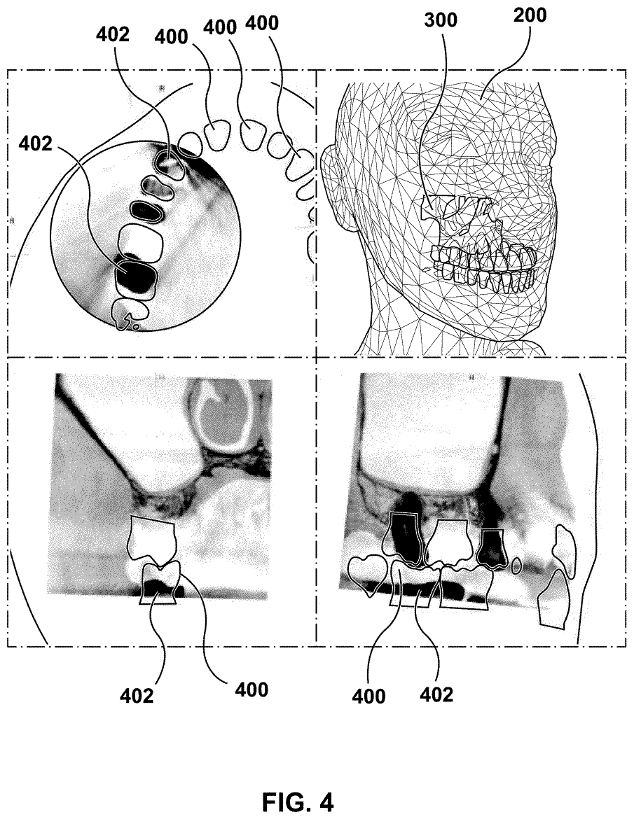

Once the patient-specific volumetric imaging data 300 of the anatomical structure of the patient is uploaded to the user interface, it is imposed or aligned with the three-dimensional reference model 200 as shown in FIG. 4. FIG. 4 illustrates volumetric image data 300 of a craniodental portion of a patient positioned in relation to the three-dimensional reference model 200 of FIG. 2. Positioning of the patient-specific volumetric imaging data 300 relative to the three-dimensional reference model 200 may be done manually, using suitable software or by other suitable means whether manual or automated.

Once the patient-specific volumetric imaging data 300 is aligned with the three-dimensional reference model 200, at least a portion of the three-dimensional reference model 200 may be deformed as at step 106 to achieve tighter alignment with at least a portion of the anatomical structure represented by the volumetric imaging data 300. Deformation of the model 200 may include repositioning or relocation of one or more of the faces 202, edges 204 or vertices 206 making up the three-dimensional reference model 200. This process is illustrated in the second, third and fourth quadrants of FIG. 4 which show, in different views and for example, the three-dimensional reference positions 400 of specific teeth according to the three-dimensional reference model 200 and the actual position of the 402 teeth of the patient as shown by the patient-specific volumetric imaging data 300. Accordingly, the positioning of the patient-specific volumetric imaging data 300, when imposed on the three-dimensional reference model 200, illustrates the relative positioning of certain patient-specific anatomical structures relative to their reference positions in the three-dimensional reference model 200. The process of deformation to bring the reference model 200 into conformity with the patient-specific volumetric imaging data 300 serves as a basis for mapping or normalization of patient-specific volumetric imaging data 300 relative to the three-dimensional reference model 200.

The result of this process is to provide a three-dimensional milieu which contains the patient-specific data suspended in a standardized position relative to the three-dimensional reference model. Once this is achieved, the patient-specific data may be related to clinically relevant anatomic positions or points of interest on the scaffolding. This may serve as a basis for making a diagnosis, determining a treatment plan or evaluating an ongoing treatment plan.

Moreover, this process may be repeated for patient-specific data associated with any number of patients. Thereby, patient-specific radiographic findings with specific anatomic positions can be associated or “mapped” with appropriate and anatomically relevant diagnostic or treatment options. Due to the standardized three-dimensional reference model 200, patterns in patient-specific data 300 can be learned by machine learning. Such patterns may include, by way of non-limiting example, rotation, orientation, number, position, relative position, relative number or any other suitable relationship between clinically relevant points of interest. Once a machine learning model is trained by using the normalized patient data in a machine learning algorithm as described, for example, at steps 110, 112 and 114, above, there is provided a means by which volumetric patient-specific data input into the model, such as at step 116, may automatically produce at least one diagnosis or at least one treatment plan specific to that patient as shown at steps 118 and 120. Moreover, this may be performed without the need for extensive expert analysis and annotation. Where a single diagnosis or treatment plan may not be suggested by the method, then the method may serve to reduce the number of statistically improbable diagnoses or treatment plans having a low probability of success from the body of results thereby automatically eliminating much of the time-consuming work typically performed by a skilled expert. Such a model would account for patient-specific anatomical nuances between the same anatomical structures of any number of patients. Such patient-specific anatomical nuances for the same anatomical structures might include differences in in size or dimensions from patient to patient for the same anatomical structure.

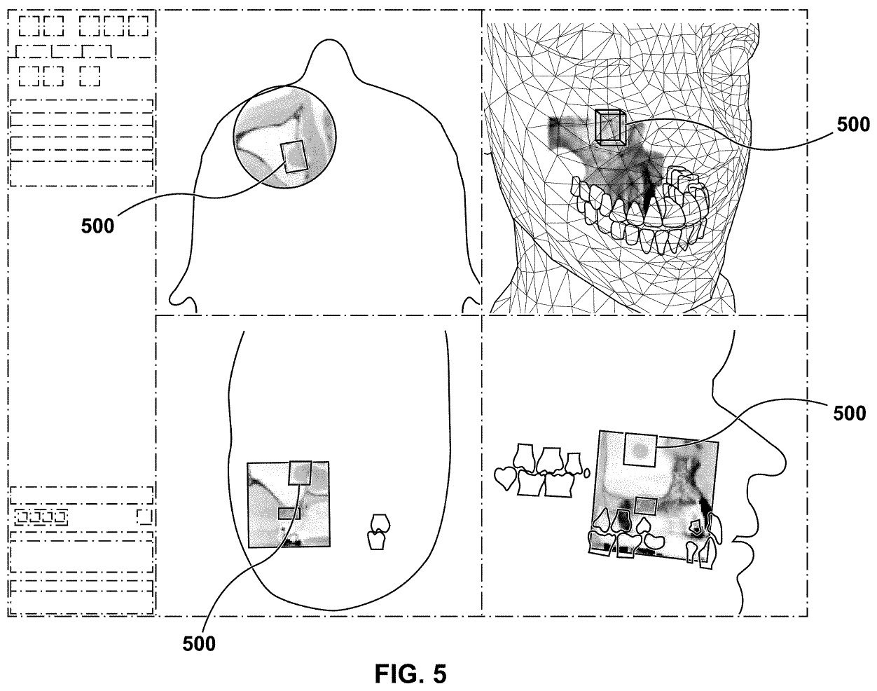

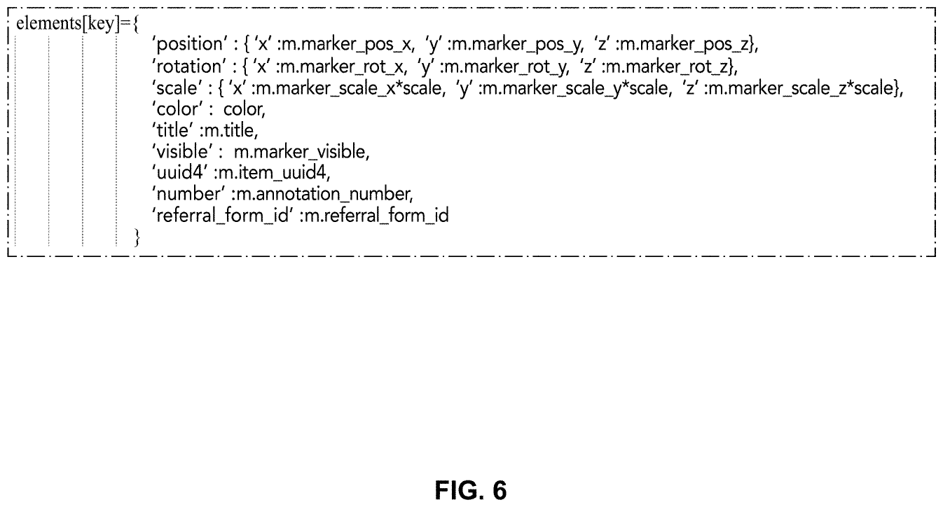

Moreover, annotations made to the patient-specific volumetric data 300 may also be mapped to specific treatment options and diagnoses. As shown in FIG. 5, annotations 500 may be made to the patient-specific volumetric data 300. Annotations are typically entered by a skilled expert manually using suitable software. Annotations may be imposed on or adjacent to one or more points of clinical relevance to serve simply as an indicator to draw the attention of a clinician during analysis or diagnosis or to be mapped for the purpose of determination of treatment options or diagnosis. However, annotations preferably include information concerning the size, direction and orientation of the point of interest. A sample data structure of an annotation is shown in FIG. 6 and includes data relating to a number of attributes of the annotation, including position, rotation and scale, among others.

As shown in FIG. 5, the annotation may take the form of a box bounding a clinical point of interest. Where the clinical point of interest is a lesion, for example, the box provides information concerning not only the width, depth and length of the lesion, but also its orientation in space and potentially its position relative to other lesions, clinical points of interest or anatomical structures. The annotations, like other clinically relevant points of interest, may be located within the three-dimensional milieu by way of the relationship with the three-dimensional reference model established by fitting the patient-specific volumetric imaging information with the three-dimensional reference model and therefore may also be evaluated by the machine learning model to be associated with treatment options and diagnoses that are suitable for the patient-specific data. Thereby, the annotations may be used as training data or input data for a machine learning model to associate the annotation information with one or more suitable diagnoses or treatment plans.

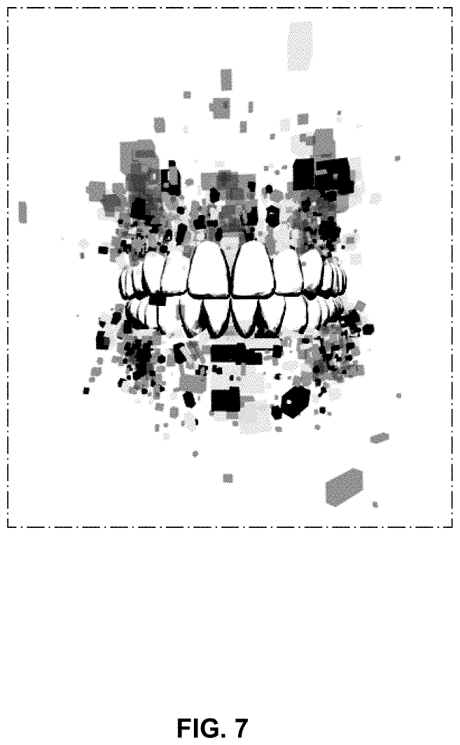

FIG. 7 illustrates a three-dimensional visualization of a plurality of annotations located relative to the three-dimensional reference model. As indicated above, annotations can be sourced from patient-specific data associated with any number of patients and normalized relative to the reference model. As more annotations are added relative to the reference model, the annotations increase in density relative to the model and form patterns relative to anatomic landmarks. Accordingly, there is provided an anatomically-normalized three-dimensional “atlas” of annotations which may be relied upon to suggest or evaluate diagnoses or treatment plans.

It should be further understood that although the annotations are shown as a rectangular box in FIG. 5, the annotations may be of any suitable shape. Preferably, the annotation is a perfect representation of the boundaries of the clinical point of interest since this would provide the most accurate information for determination of a diagnosis or treatment plan as well as the most accurate information for a machine learning system. However, such a level of detail may not meet the time constraints demanded by clinical analysis and diagnosis. Accordingly, a less detailed approximation of the boundaries of the point of interest may be preferred. Accordingly, the annotation may take any shape which may provide such an approximation.

The annotations provide further advantage in that the machine learning model can be trained to recognize patterns in the annotations to serve as the basis for suggesting a diagnosis or treatment plan. In processing of digital images, including digital radiographic images, the images are typically analyzed on a pixel-by-pixel basis or in groups of pixels in order to identify patterns and make a probabilistic identification of an anatomical structure or condition. This detailed type of analysis requires a high level of computer processing. In the present method, the information associated with the annotations may serve as the basis for pattern recognition. The system may associate patient-specific annotation information with annotation information in the standardized model. Accordingly, the patient-specific annotation may be associated with diagnoses or treatment options using the machine learning model. Since the relevant structure (i.e. the annotation) is identified to the model, it is not required to first perform pixel-by-pixel pattern recognition. Thereby, the processing demands for automatically generating a diagnosis or treatment option are reduced.

A system of one or more computers or computerized architectural components can be configured to perform particular operations or actions by virtue of having software, firmware, hardware, or a combination thereof installed on the system that in operation causes or cause the system to perform the actions. One or more computer programs recorded on one or more computer storage devices can be configured to perform particular operations or actions by virtue of including instructions that, when executed by data processing apparatus, cause the apparatus to perform the actions automatically and/or in real-time.

While the invention has been described in terms of specific embodiments, it is apparent that other forms could be adopted by one skilled in the art. For example, the methods described herein could be performed in a manner which differs from the embodiments described herein. The steps of the method could be performed using similar steps or steps producing the same result but which are not necessarily equivalent to the steps described herein. Some steps may also be performed in different order to obtain the same result. Similarly, the apparatuses and systems described herein could differ in appearance and construction from the embodiments described herein, the functions of each component of the system could be performed by components of different construction but capable of a similar though not necessarily equivalent function, and appropriate materials could be substituted for those noted. Accordingly, it should be understood that the invention is not limited to the specific embodiments described herein. It should also be understood that the phraseology and terminology employed above are for the purpose of disclosing the illustrated embodiments, and do not necessarily serve as limitations to the scope of the invention.

What is claimed is:

1. A method for mapping patient-specific volumetric imaging data relative to a three-dimensional reference model comprising the steps of:
acquiring volumetric imaging data of an anatomical structure of at least one patient;
imposing the volumetric imaging data of the anatomical structure to the three-dimensional reference model of the anatomical structure; and,
deforming at least a portion of the three-dimensional reference model to conform at least approximately with the volumetric imaging data representing at least a portion of the anatomical structure of the at least one patient to map volumetric imaging data representing at least a portion of the anatomical structure of the at least one patient relative to the at least a portion of the three-dimensional reference model.

2. The method of claim 1, wherein the three-dimensional reference model includes a plurality of vertices, and the step of deforming at least a portion of the three-dimensional reference model further includes the step of:
changing a position of at least one of the plurality of vertices.

3. The method of claim 1, wherein the patient-specific volumetric imaging data further includes at least one annotation, the method further comprising the step of:
mapping a position of the annotation relative to the at least a portion of the three-dimensional reference model.

4. The method of claim 1, wherein the at least one patient includes a plurality of patients.

5. The method of claim 1, wherein the anatomical structure of the at least one patient is a craniodental structure.

6. The method of claim 1, further comprising the step of:
using the mapped volumetric imaging data of the at least a portion of the anatomical structure as at least one of training data and input data for a model.

7. The method of claim 1, further comprising the step of:
determining at least one of a diagnosis and a treatment plan based on the mapped volumetric imaging data.

8. The method of claim 1, further comprising the step of:
using the mapped volumetric imaging data as input data for a machine learning algorithm for training a model.

9. The method of claim 8, wherein the model is for diagnosing at least one condition of the at least one patient based on the patient-specific mapped volumetric imaging data.

10. The method of claim 8, wherein the model is for at least one of determining and evaluating at least one patient treatment plan based on the patient-specific mapped imaging data.

11. The method of claim 4 further comprising the step of:
using the mapped volumetric imaging data as training data for a machine learning algorithm to train a model.

12. The method of claim 11, wherein the model is for diagnosing at least one condition of the at least one patient based on the patient-specific mapped volumetric imaging data.

13. The method of claim 11, wherein the model is for at least one of determining and evaluating at least one patient treatment plan based on the patient-specific mapped volumetric imaging data.

14. The method of claim 3, wherein the annotation is a box bounding a point of interest in the volumetric imaging data of the anatomical structure of the at least one patient.

15. The method of claim 14, wherein the box has a width, a depth, a length, a position, and an orientation.

16. The method of claim 15, further comprising the step of:
mapping at least one of the width, the depth, the length, the position and the orientation of the annotation relative to the at least a portion of the three-dimensional reference model.

17. A system for mapping patient-specific volumetric imaging data relative to a three-dimensional reference model comprising:
an imaging device for acquiring volumetric imaging data of an anatomical structure of at least one patient;
means for imposing the volumetric imaging data of the anatomical structure to the three-dimensional reference model of the anatomical structure; and,
means for deforming at least a portion of the three-dimensional reference model to conform at least approximately with the volumetric imaging data representing at least a portion of the anatomical structure of the at least one patient to map the volumetric imaging data representing at least a portion of the anatomical structure of the at least one patient relative to the at least a portion of the three-dimensional reference model.

18. The system of claim 17, wherein the three-dimensional reference model includes a plurality of vertices, and the step of deforming at least a portion of the three-dimensional reference model further includes:
means for changing the position of at least one of the plurality of vertices.

19. The system of claim 17, wherein the patient-specific volumetric imaging data further includes at least one annotation, the system further comprising:
means for mapping a position of the annotation relative to the at least a portion of the three-dimensional reference model.

20. The system of claim 17, wherein the at least one patient includes a plurality of patients.

21. The system of claim 17, wherein the anatomical structure of the at least one patient is a craniodental structure.

22. The system of claim 17, further comprising:
means for using mapped volumetric imaging data of the at least a portion of the anatomical structure as at least one of training data and input data for a model.

23. The system of claim 17, further comprising:
means for determining at least one of a diagnosis and a treatment plan based on the normalized volumetric imaging data.

24. The system of claim 17, further comprising:
means for using the normalized volumetric imaging data as input data for a machine learning algorithm for training a model.

25. The system of claim 24, wherein the model is for diagnosing at least one condition of the at least one patient based on the patient-specific mapped volumetric imaging data.

26. The system of claim 24, wherein the model is for at least one of determining and evaluating at least one patient treatment plan based on the patient-specific mapped imaging data.

27. The system of claim 20 further comprising:
means for using the mapped volumetric imaging data as training data for a machine learning algorithm to train a model.

28. The system of claim 24, wherein the model is for diagnosing at least one condition of the at least one patient based on the patient-specific mapped volumetric imaging data.

29. The system of claim 27, wherein the model is for at least one of determining and evaluating at least one patient treatment plan based on the patient-specific mapped volumetric imaging data.

30. The system of claim 19, wherein the annotation is a box bounding a point of interest in the volumetric imaging data of the anatomical structure of the at least one patient.

31. The system of claim 30, wherein the box has a width, a depth, a length, a position, and an orientation.

32. The system of claim 31, further comprising:
means for mapping at least one of the width, the depth, the length, the position and the orientation of the annotation relative to the at least a portion of the three-dimensional reference model.
